(19) United States
(12) Patent Application Publication
(10) Pub. No.: US 2023/0210471 A1
(43) Pub. Date: Jul. 6, 2023

(54) Assessment of Hemodynamics Parameters

(71) Applicant: SILVERLEAF MEDICAL SCIENCES INC., Chino, CA (US)

(72) Inventors: Jianwei (Arnold) Zheng, Chino, CA (US); Islam Abudayyeh, Redlands, CA (US); Wenzan Zhang, Irvine, CA (US)

(73) Assignee: SILVERLEAF MEDICAL SCIENCES INC., Chino, CA (US)

(21) Appl. No.: 17/566,721

(22) Filed: Dec. 31, 2021

(51) Int. Cl. A61B 5/00; A61B 5/0205

(52) U.S. Cl. CPC A61B 5/7275; A61B 5/7267; A61B 5/0205; A61B 5/021

(57) ABSTRACT

The present disclosure relates to an apparatus for predicting a hemodynamics parameter being conventionally obtained from an implanted sensor or catheter (invasive sensor), e.g., pulmonary artery pressure, based on noninvasive biosignals, such as electrocardiographic (ECG), impedance cardio graphic (ICG), phonocardiogram (PCG), pulse oximetry plethysmograph (PPG). The present disclosure also relates to a method of feeding multiple noninvasive biosignals and/or general inputs into an AI model or AI models to predict a hemodynamics parameter, such as pulmonary artery pressure, which is conventionally obtained from an implanted sensor or catheter.

### BACKGROUND

Hemodynamics are the mechanisms through which blood circulates through the body. Specifically, hemodynamics is a term used to describe the intravascular and intracardiac pressures and flow that occur during cardiac cycles. The vascular system is typically a closed circuit. Pressure and flow variations in the venous system will necessarily affect the arterial system and vice versa. Therefore, hemodynamic measurements are not static values but, rather, vary beat to beat depending on the pressure and flow that occur within and between the arterial and venous compartments. The decision to treat a hemodynamic value should always be taken with full consideration of the overall physiology, underlying conditions, and goals of care.

Hemodynamic monitoring can reveal changes in cardiovascular function, and the interpretation of such changes may prompt therapeutic interventions. Multiple assessment methods for hemodynamic status, conducted by either invasive or noninvasive devices, are available. These report pulmonary arterial pressure (PAP), pulmonary arterial widget pressure (PAWP), arterial blood pressure (ABP), central venous pressure (CVP), electrocardiographic (ECG), impedance cardio graphic (ICG), phonocardiogram (PCG), pulse oximetry plethysmograph (PPG), peripheral venous pressure waveform (PVPW), peripheral arterial pressure waveform (PAPW), cardiac output (CO), stroke volume (SV), and left ventricular ejection fraction (LVEF).

Properly interpreting the hemodynamic measurements can help detect adverse conditions earlier. For example, increased CO may indicate a high flow state, decreased peripheral vascular resistance or shunting while a decreased CO may indicate a decrease in circulating volume, a reduced left ventricular contractile function, or valvular disease. CVP readings are used to assess volume status and approximate right ventricular end diastolic pressure (RVEDP). Low CVP values typically reflect hypovolemia or decreased venous return; high CVP values reflect volume overload, increased venous return or right sided cardiac failure. ABP may be affected by changes in the cardiac output (CO) and systemic vascular resistance (SVR) and reflects the arterial pressure in the vessels perfusing the organs; a low ABP indicates decreased blood flow through the organs; a high ABP indicates an increased cardiac workload or higher systemic vascular resistance. An increased PAP indicates increased pulmonary artery hypertension, which may be due to COPD, emphysema, pulmonary embolus, pulmonary edema, left ventricular failure, along with many other etiologies. Wedge pressure (PCWP) is used to approximate left atrial pressures as a balloon-tipped catheter wedged in the pulmonary artery creates a static fluid-filled conduit to the pulmonary veins. High PCWP may indicate left ventricle failure, mitral valve pathology, diastolic dysfunction with increased filling pressures, or increased pericardial pressure due to tamponade physiology. A decreased SV may indicate impaired cardiac contractility or valve dysfunction and may result in heart failure, while an increased SV may be caused by an increase in circulating volume or an increase in inotropy, such as with hypertrophic cardiomyopathy.

However, PAP, PAWP, and CVP are typically only reliably measured by invasive means. ABP can be measured by intra-arterial catheters. Some noninvasive methods can estimate ABP, but catheter detection is more reliable and accurate. The measurement of SV and CO have a similar situation with ABP. LVEF can be calculated through an echocardiogram, magnetic resonance image, or nuclear medicine scan. ECG, PPG, PCG, ICG, respiration impedance waveform, PVPW, and PAPW can be obtained by noninvasive techniques.

### SUMMARY OF THE INVENTION

In one aspect, the present invention provides an apparatus for predicting a hemodynamics parameter being conventionally obtained from an implanted sensor or catheter. The apparatus comprises: a processor configured to perform a computer program to choose a qualified waveform record of a noninvasive input variable being collected by a noninvasive sensor or detector; analyze the waveform record with an AI model; and predict the hemodynamics parameter.

In another aspect, the present invention provides a computer implemented method of predicting a hemodynamics parameter being conventionally obtained from an implanted sensor or catheter apparatus in the mammal. The method comprises choosing a qualified waveform record of a noninvasive input variable of a mammal being collected by a noninvasive sensor or detector; analyzing the waveform record with an AI model; and predicting the hemodynamic parameter.

In certain embodiments, the noninvasive input variable is selected from a noninvasive biosignal/a noninvasive hemodynamics parameter, a general input variable, or a combination thereof.

In further embodiments, the noninvasive input variable contains at least two noninvasive biosignals or noninvasive hemodynamics parameters selected from a group consisting of electrocardiographic (ECG), impedance cardio graphic (ICG), phonocardiogram (PCG), pulse oximetry plethysmograph (PPG), peripheral venous pressure waveform (PVPW), peripheral arterial pressure waveform (PAPW), respiration waveform (RESP), echocardiogram, airway resistance, blood sugar level waveform, and a combination thereof.

In certain embodiments, the general input is selected from the group consisting of null, age, gender, body mass index (BMI), temperature, motion status, and a combination thereof.

In certain embodiments, the AI model is a base regression model, Bagging regressor, AdaBoost regressor, voting regressor, or a combination thereof.

In further embodiments, the base regression model is selected from a group consisting of Decision Tree (DT), K Nearest Neighbors (KNN), Nearest Centroid (NC), Gaussian Naive Bayesian (GNB), Multinomial Naive Bayesian (MNB), Complement Naive Bayesian (CNB), Bernoulli Naive Bayesian (BNB), General Linear Regression (GLR), Quadratic Discriminant Analysis (QDA), Multinomial Logistic Regression (MLR), Multi-layer Perceptron Neural Net (MPN), Ridge Regression (RR), Linear Regression with Stochastic Gradient Descent (LCSGD), Passive Aggressive Regression (PAC), Linear SVC (SVC), Random Forest (RF), Extremely Randomized Trees (ERT), Gradient Boosting Tree (GBT), Extreme Gradient Boosting Tree (EGBT), convolutional neural network (CNN) with residual structure, long-term and short-term memory (LSTM) recurrent neural network, double direction LSTM, CNN with residual block and transformer structure, and a combination thereof.

In further embodiments, Bagging regressor is a meta-regressor and uses all of the base regression models.

In further embodiments, AdaBoost regressor is a meta-estimator and utilizes the base models of DT, GNB, MNB, CNB, BNB, MLR, RR, LCSGD, and SVC.

In further embodiments, Voting regressor, if present, is a meta-estimator and uses the base models of DT, KNN, GNB, MNG, CNB, BNB, GLR, MLR, QDA, RR, LCSGD, PAC, MPN, SVC, RF, ERT, and GBT.

In some embodiments, the AI model is convolutional neural network (CNN) with residual structure, long-term and short-term memory (LSTM) recurrent neural network, double direction LSTM, CNN with residual block and transformer structure, EGBT, or a combination thereof.

In certain embodiments, the hemodynamics parameter predicated in these aspects is selected from a group consisting of pulmonary arterial pressure (PAP), pulmonary arterial widget pressure (PAWP), arterial blood pressure (ABP), central venous pressure (CVP), right atrial pressure (RAP), right ventricular pressure (RVP), cardiac output (CO), stroke volume (SV), left ventricular ejection fraction (LVEF), and a combination thereof.

In further embodiments, the hemodynamics parameter predicated in these aspects is pulmonary artery pressure (PAP).

### BRIEF DESCRIPTION OF DRAWINGS

FIG. 1 depicts a system level block diagram.

FIG. 2 depicts an ECG and respiration impedance module diagram.

FIG. 3 depicts an ICG module diagram.

FIG. 4 depicts a PPG module diagram.

FIG. 5 depicts a PAWP and PVWP diagram.

FIG. 6 depicts a PCG module diagram.

FIG. 7 depicts a central data processing box diagram.

FIG. 8 depicts signal synthesize module workflow.

FIG. 9 depicts the overlapping sliding window method.

FIG. 10 depicts a three-band PPG collection.

FIG. 11 depicts a comparison design. Each model is configured by a set of learning parameters and fed by features, either raw signal or wavelet scattering features that were generated from waveform records with different window sizes and step lengths.

FIG. 12A depicts six signals and segmentation method; in the figure ABP, CVP, respiration, PPG, ECG are input predictors, and PAP is the outcome variable.

FIG. 12B depicts segmentation method (the sliding window method), which was adopted to generate input samples.

FIG. 13A depicts wavelet scattering transform and wavelet decomposition: the waveforms constructed by different level wavelet coefficients.

FIG. 13B depicts wavelet scattering transform and wavelet decomposition: the power spectrum of 4 level wavelet scattering transformation coefficients.

FIG. 14 depicts neural network architecture. The resNet50 design structure has 16 residual blocks, presenting 50 convolutional layers.

FIG. 15 depicts baseline characteristics for all patients enrolled study. (A) Frequency distribution of the first admitted ICU unit. (B) Ethnicity distribution (C) Religion distributions (D) Marital status distributions (E) Language distribution (F) Insurance distribution (G) ICD-9 code distribution.

FIG. 16 depicts prediction result and residual analysis. A segment of observational PAP signal compared with prediction ones given by AI model.

FIG. 17 depicts five phases of AI model optimization.

### DETAILED DESCRIPTION OF INVENTION

The instant disclosure provides an apparatus and a method to predict a subject's hemodynamics parameter, which is normally obtained from an implanted sensor in the subject using input biosignals, which can be easily obtained from noninvasive sensors attached on a subject's skin (e.g., human skin), particularly in the chest area.

In one embodiment, the apparatus and the method use a built-in AI model and noninvasive input biosignals (e.g., hemodynamics parameters collected by noninvasive sensors) and/or other general inputs to predict the aforementioned hemodynamics parameters.

In one embodiment, noninvasive biosignals include at least electrocardiographic (ECG), impedance cardiograph is (ICG), phonocardiogram (PCG), pulse oximetry plethysmograph (PPG), peripheral venous pressure waveform (PVPW), peripheral arterial pressure waveform (PAPW), respiration waveform. (RESP), echocardiogram, airway resistance, blood sugar level waveform and a combination thereof. The general inputs include at least age, gender, body mass index (BM), temperature, motion status, and a combination thereof. In a further embodiment, at least two noninvasive biosignals are used to predict the hemodynamics parameter.

In one embodiment, the hemodynamics parameter, which can be predicted using the apparatus and the method, includes at least pulmonary arterial pressure (PAP), pulmonary arterial widget pressure (PAWP), arterial blood pressure (ABP), central venous pressure (CVP), right atrial pressure (RAP), right ventricular pressure (RVP), cardiac output (CO), stroke volume (SV), left ventricular ejection fraction (LVEF), and a combination thereof.

In another embodiment, examples of AI models include base regression models, Bagging regressor, AdaBoost regressor, voting regressor, and a combination thereof. Examples of base regression models include Decision Tree (DT), K Nearest Neighbors (KNN), Nearest Centroid (NC), Gaussian Naive Bayesian (GNB), Multinomial Naive Bayesian (MNB), Complement Naive Bayesian (CNB), Bernoulli Naive Bayesian (BNB), General Linear Regression (GLR), Quadratic Discriminant Analysis (QDA), Multinomial Logistic Regression (MLR), Multi-layer Perceptron Neural Net (MPN), Ridge Regression (RR), Linear Regression with Stochastic Gradient Descent (LCSGD), Passive Aggressive Regression (PAC), Linear SVC (SVC), Random Forest (RF), Extremely Randomized Trees (ERT), Gradient Boosting Tree (GBT), Extreme Gradient Boosting Tree (EGBT), convolutional neural network (CNN) with residual structure, long-term and short-term memory (LSTM) recurrent neural network, double direction LSTM, CNN with residual block and transformer structure, and a combination thereof. Bagging regressor is a meta-regressor and uses all of the above enumerated base regression models. AdaBoost regressor is a meta-estimator, which utilizes the following base regression models: DT, GNB, MNB, CNB, BNB, MLR, RR, LCSGD, and SVC. Voting regressor is another meta-estimator, which uses the following base regression models: DT, KNN, GNB, MNG, CNB, BNB, GLR, MLR, QDA, RR, LCSGD, PAC, MPN, SVC, RF, ERT, and GBT.

In a further embodiment, at least the following models demonstrate superior performance: CNN with residual structure, CNN with residual block and transformer structure, LSTM, double direction LSTM, and EGBT. In a further embodiment, CNN with residual structure attains the highest performance measurement score.

In another embodiment, clinical-grade level accuracy of various AI models was evaluated according to the following procedure. Since the AI model's performance depends on the input features and learning parameters, a wide-scale comparison was performed to scout out the best AI model with optimal input features and learning parameters. For instance, a training-validation-testing design was used to perform such a large-scale comparison. The model comparison and optimization consist of five phases (presented in the central illustration figure): 1) data processing phase to choose qualified waveform record and carry out noise reduction; 2) data preparation phase to fragment the waveform record to multiple sample windows; 3) feature extraction phase where wavelet scattering transform method was studied; 4) model tunning and comparison phase to find the optimal learning parameters and input features; 5) evaluation phase to evaluate, interpret and report the model performance. In a further embodiment, all the aforementioned models were trained and tested using the same scheme mentioned above, and R² scores were compared to choose the optimal model.

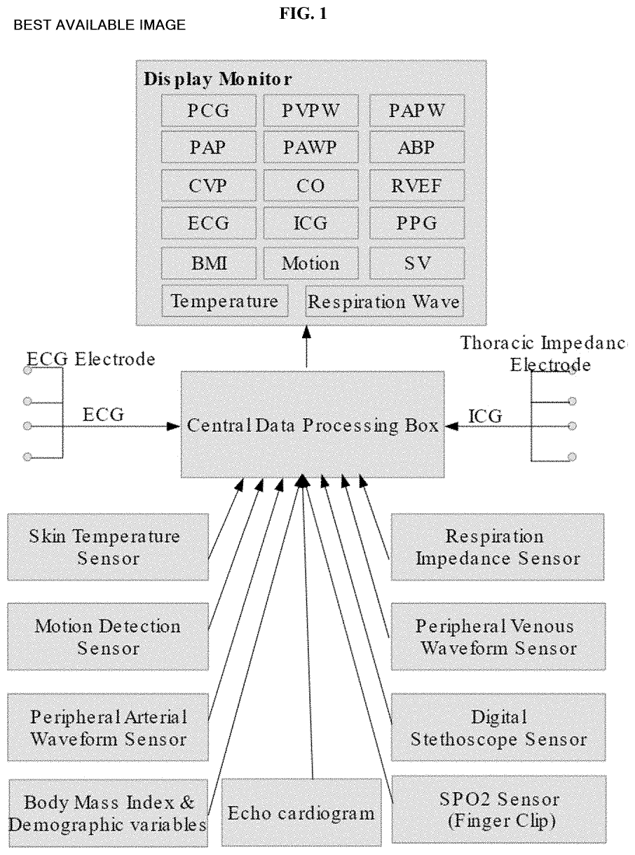

In a further embodiment, the current disclosure provides a central processing box (FIG. 1) which collects signals from at least two of electrocardiographic, thoracic impedance, respiration impedance, digital stethoscope, pulse oximetry, peripheral venous waveform, peripheral arterial waveform, skin temperature, and motion. The output of the box includes ECG, ICG, Respiration waveform, PCG, PPG, BMI, PVPW, PAPW, Temperature, Motion status, PAP, ABP, PAWP, CVP, CO, SV, LVEF and a combination thereof. Among these outputs, ECG, ICG, Respiration waveform, PCG, PPG, BMI, PVPW, PAPW, temperature, and motion parameters are directly obtained from the associated sensors after signal processing. PAP, ABP, PAWP, CVP, CO, SV, and LVEF are achieved by post-processing utilizing an artificial intelligence digital signal chip. The completed system diagram is depicted in FIG. 1.

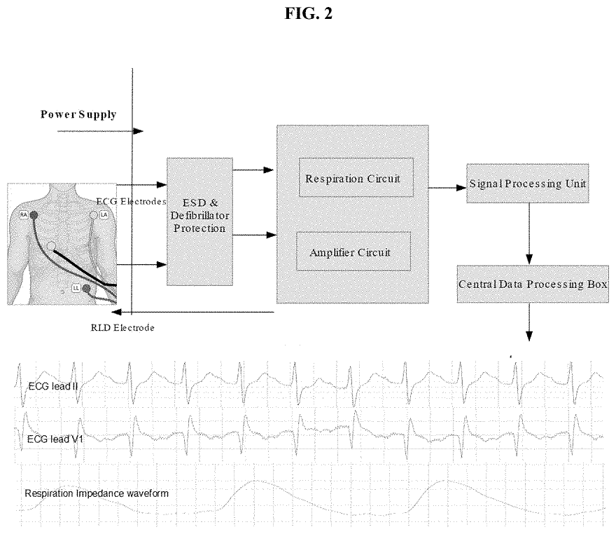

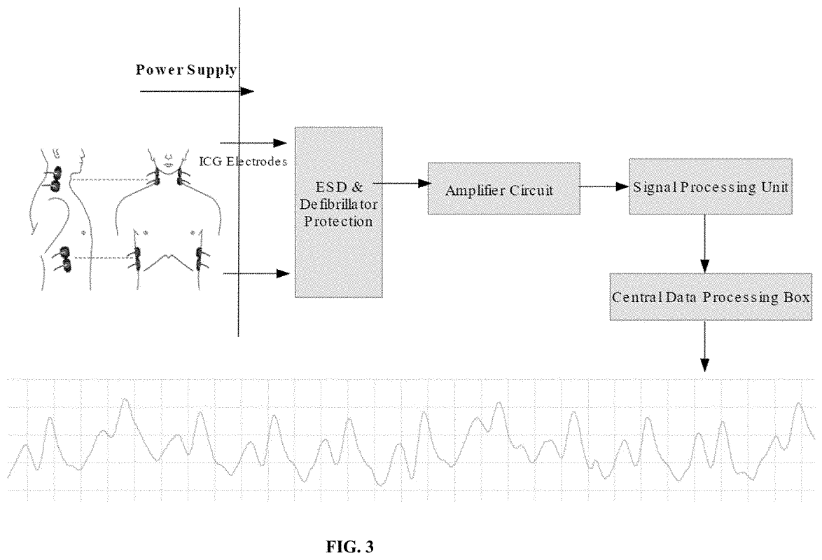

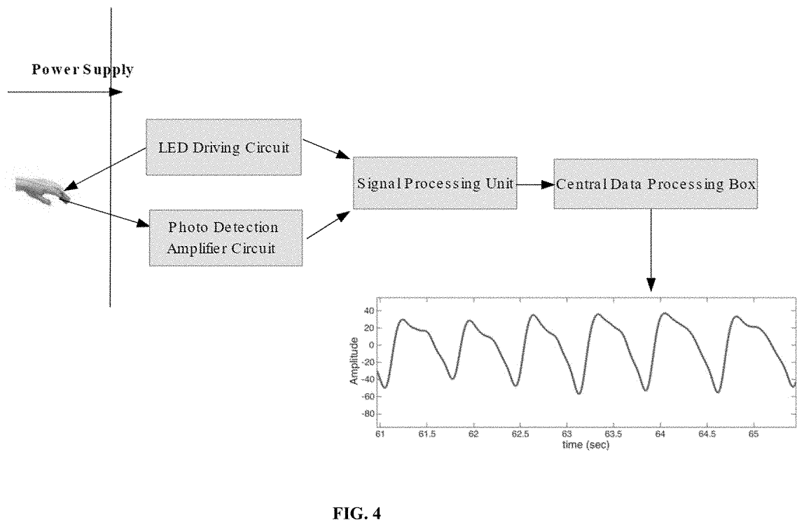

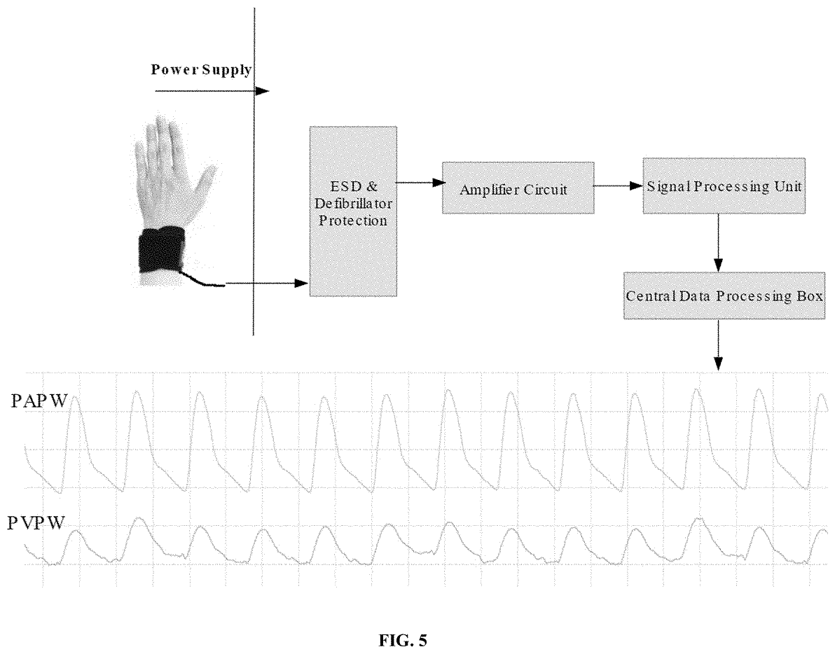

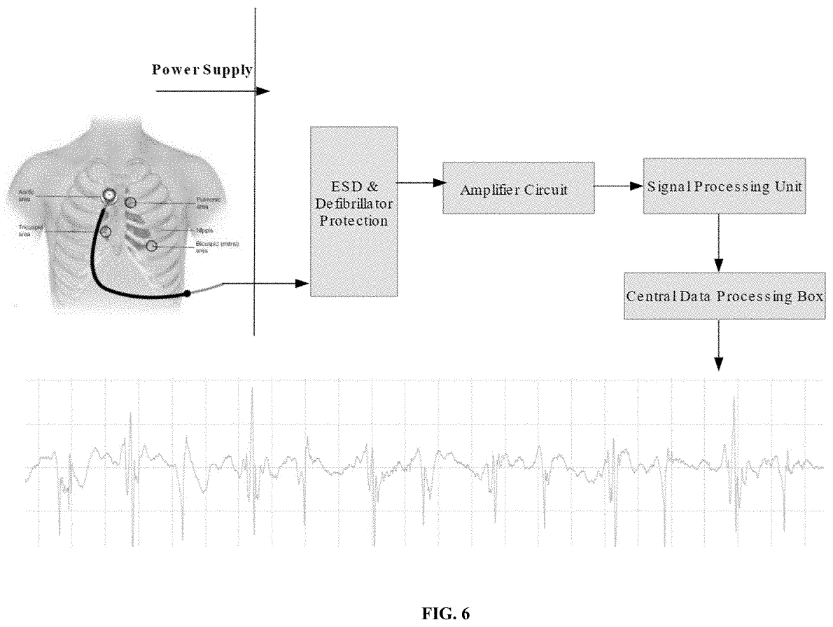

In a further embodiment, the current disclosure provides examples of sensors and subsystems of the apparatus of this aspect. ECG and Respiration impedance Module is depicted in FIG. 2. ECG module can take 3-6 channels input, and respiration impedance signal can be collected from ECG electrodes. ICG module is illustrated in FIG. 3. ICG module can take 4 pairs of electrode input. Bio-impedance analysis and bio impedance spectroscopy methods are employed to attain thoracic impedance signal. The computation to generate ICG can be done by the signal processing unit. PPG and SPO2 modules are depicted in FIG. 4. PAWP and PVWP modules are depicted in FIG. 5. Two piezo-electric sensors were placed on the skin over the radial vein and artery on the volar aspect of the wrist. When placed directly over these peripheral veins and arteries, the sensors can detect small deflections in the skin overlying the vein and artery that occur with the cardiac cycle. PCG and digital stethoscope modules are depicted in FIG. 6.

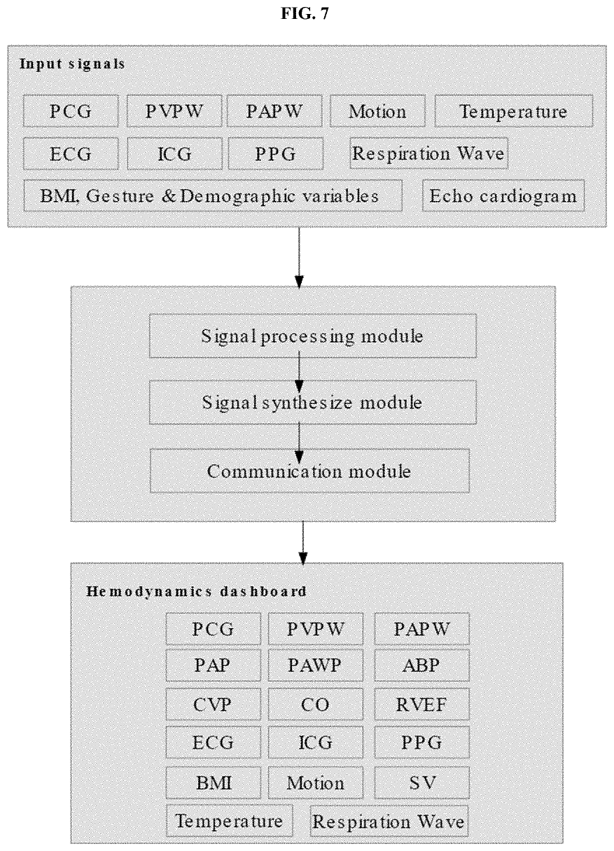

The central data processing box is depicted in FIG. 7. When input signals go through the signal processing module, it can preprocess input signals, including quality checking, noise reduction, electrode adhesion, and validation. After preprocessing, the signal synthesis module uses the pre-built artificial intelligence model to estimate PAP, PAWP, CVP, ABP, LVEF, SV, and CO. Finally, the communication module can push all signals to the hemodynamics dashboard. The communication module can push the required signal per configuration.

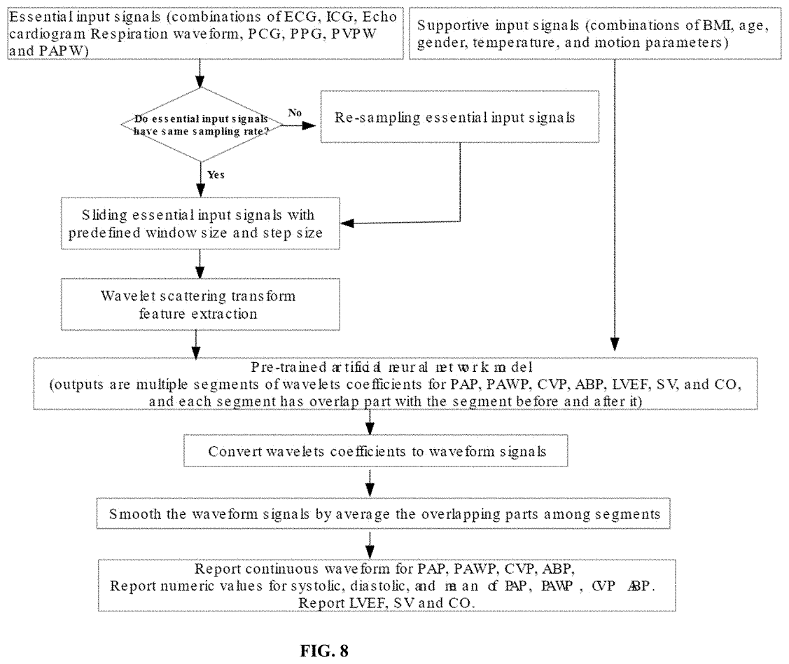

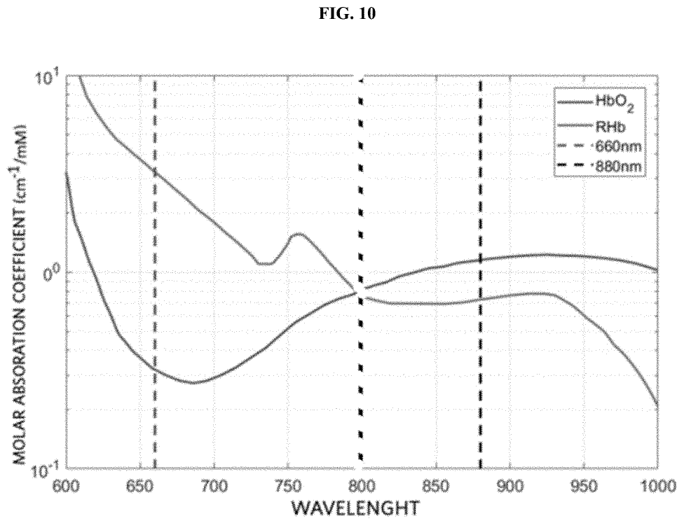

The signal synthesis module (shown in FIG. 8) is a vital component of the device box. It can use any combination of essential input signals including ECG, ICG, Respiration waveform, PCG, PPG, PVPW, and PAPW to predict PAP, ABP, PAWP, CVP, CO, SV, and LVEF, a total of 7 parameters. BMI, temperature, and motion status will provide additional information for prediction. For example, if the device box is only given ECG and ICG signals, it still can predict PAP, ABP, PAWP, CVP, CO, SV, and LVEF. If the device box is given ECG, ICG, PCG, and PPG, the prediction accuracy for the seven parameters mentioned above will improve accordingly. The more input signals are provided, the better prediction performance is achieved. Three bandwidth PPG collection is depicted in FIG. 10.

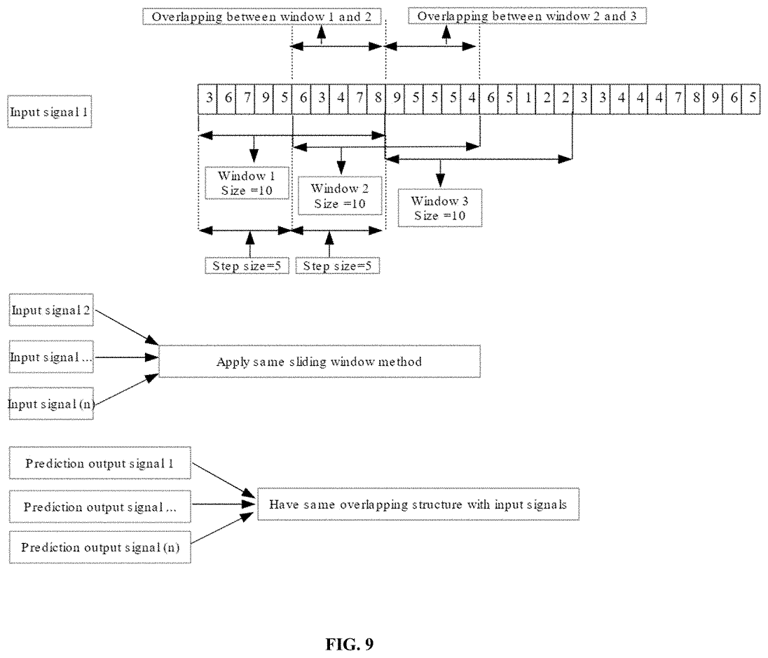

For essential input signals, the device box will unify the sampling rate. The overlapping sliding window method (shown in FIG. 9) is used to improve the prediction accuracy. The pre-trained artificial neural network model takes wavelet scattering transform features as input and provides the wavelets coefficients as output. The signal synthesis module will convert the model output to time-domain signals (waveform data). After this conversion, the average values of signals overlapping among segments (windows) are final output waveform data.

In another embodiment, the wavelet scattering and discrete wavelets analysis methods were used for prediction and smoothing signals. Specifically, a wavelet scattering feature extraction method was used to get the invariant presentation of original input signals. The coefficients of wavelet scattering are inputs of AI models. The prediction outcomes of the AI models are coefficients of discrete wavelets decomposition. The wavelets coefficients are subsequently converted back to time-domain signals. Thus, the predicted time-domain signals will be smooth waveforms. According to the settings of the overlapping sliding window method, this method smooths the predicted time-domain signals by averaging the values within overlapping parts among windows.

In another aspect, the current disclosure provides a method of synchronizing data collection from the sensors or subsystem of the apparatus for predicting a hemodynamics parameter. In one embodiment, the current disclosure provides a noninvasive multi-sensor device to collect multiple hemodynamic parameters simultaneously.

Hereinafter, the present invention will be described in more detail by way of Examples. The invention will be more readily understood by reference to the following examples. However, the technical scope of the present invention is not intended to be limited only to the following Examples.

### III. EXAMPLES

### Example 1. Experiment Designs

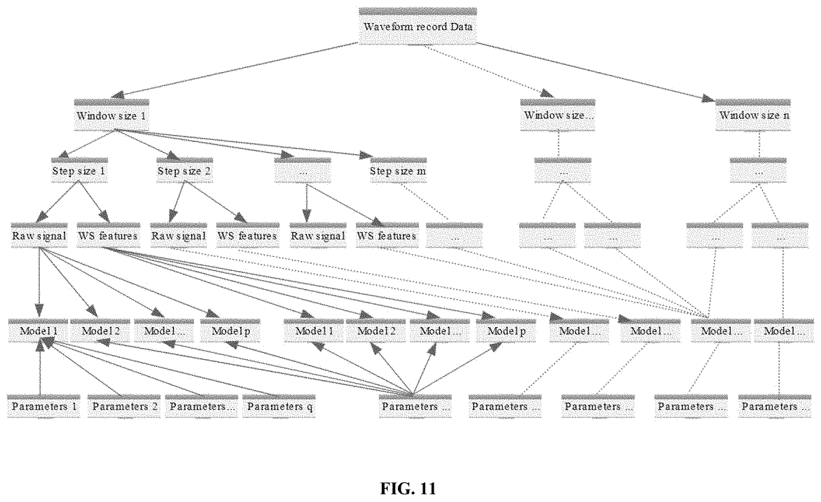

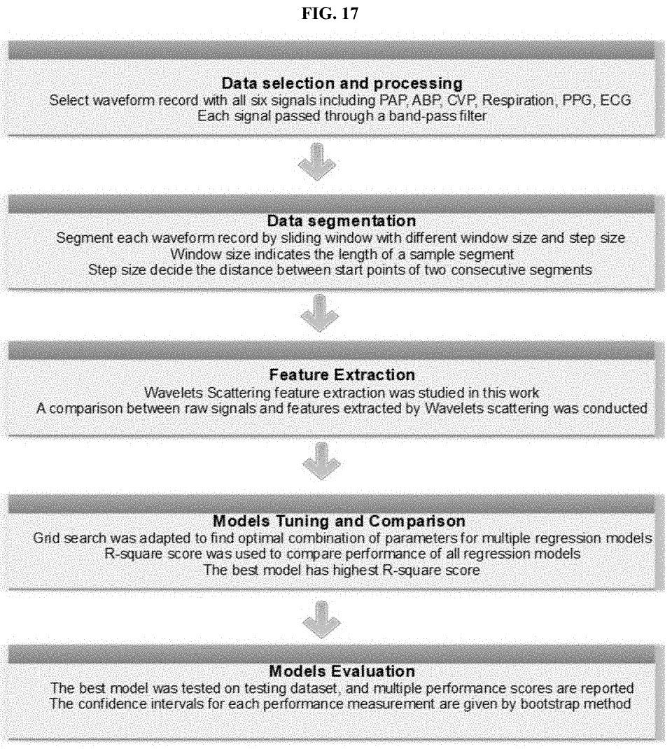

The study was conducted in accordance with the Declaration of Helsinki. This study was designed to answer one vital question: how to use various numbers of known and relatively easy to measure hemodynamics parameters in combination with AI models to predict other important hemodynamic parameters with a clinical-grade level accuracy. Since the AI model's performance depends on the input features and learning parameters, a wide-scale comparison (shown in FIG. 11.) was designed to scout out the best AI model with optimal input features and learning parameters. This study employed a training-validation-testing design to perform such a large-scale comparison. The study consists of five phases (shown in FIG. 17): 1) data processing phase to choose qualified waveform record and carry out noise reduction; 2) data preparation phase to fragment the waveform record to multiple sample windows; 3) feature extraction phase where wavelet scattering transform method was studied; 4) model tunning and comparison phase to find the optimal learning parameters and input features; 5) evaluation phase to evaluate, interpret and report the model performance. Selected AI models as enumerated in the detailed description above were trained and tested using the same scheme mentioned above, and R² scores were compared to choose the optimal model, a similar practice successfully adopted in prior work.

Patient Selection

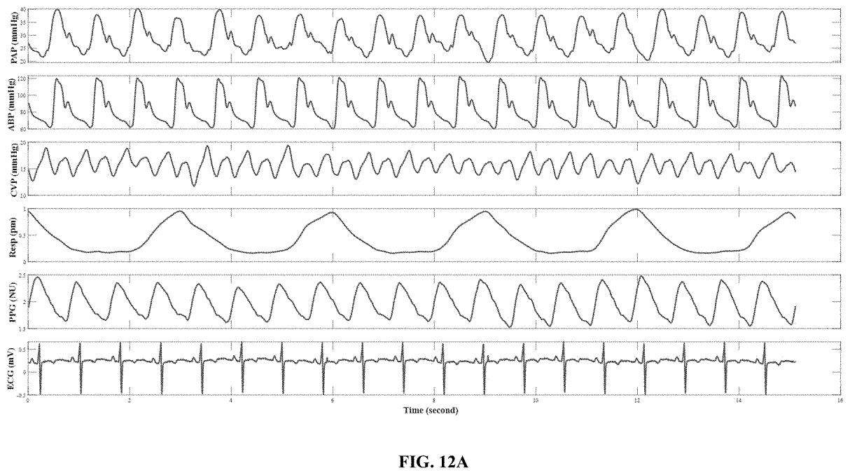

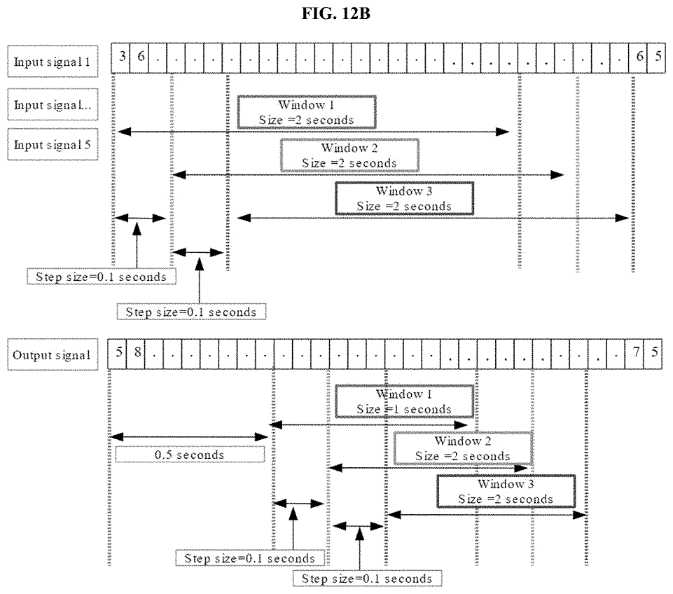

The MIMIC-III Waveform Database Matched Subset contains 22,317 waveform records from 10,282 patients admitted to the Beth Israel Deaconess Medical Center in Boston, Mass., USA. This database is a subset of the MIMIC-III Waveform Database, representing those records associated with the patients who have identified clinical notes available in the MIMIC-III Clinical Database. These recordings typically include digitized signals such as ECG, ABP, CVP, PAP, respiration, and PPG, but not every record simultaneously has these six signals. Thus, a total of 180 patients were selected from matched subset who experienced PAC procedures and had complete waveform records consisting of six signals, including PAP, ABP, CVP, respiration, PPG, and ECG lead II. An example of a waveform record segment is presented in FIG. 12A.

Data Processing Protocol and Segmentation

All or a subset of the six signals (ECG, ABP, CVP, PAP, respiration, and PPG), which were directly obtained from corresponding sensors and/or synthesized signals (ECG, ABP, CVP, PAP, respiration, and PPG), which were predicted using other known hemodynamic parameters, were synchronized to a 125 Hz sampling rate when the waveform database was digitalized. The bandpass filters consisted of highpass 50 Hz and lowpass filter 0.5 Hz were applied on waveform records from 180 patients. In this example, the sliding window method was employed to generate model input samples. A window with a specified length moves over the waveform record, step by step, and data values within the window composed input samples to wavelets scattering transformation feature extraction method. A great scale competition was deployed based on the performance of AI models to find the optimal window and step length. The searching values of window size range from about 0.05 seconds to about 50 seconds with a space of from about 0.05 seconds to about 5 seconds. The searching values of step size range from about 0.05 seconds to the value of the window size with a span from about 0.01 seconds to about 1 second. In a preferred combination, the searching values of window size range from 0.5 to 5 seconds with a space of 0.5 seconds, and those of step size range from 0.1 seconds to the value of window size with a span of 0.1 seconds.

Wavelet Scattering Network and Wavelet Analysis

Fourier analysis was used to reveal the frequency domain information. However, it cannot accurately track frequency change precisely aligned with time-domain even though fast Fourier transform and windowed Fourier transform were developed to tackle this problem. Wavelet transform addresses this problem and can present frequency distribution on any time scale. Moreover, the wavelet scattering network proposed by Mallet was developed to present frequency spectrum on multiscale contractions. The more essential characteristics of wavelet scattering transform favored by the AI models are the linearization of hierarchical symmetries and sparse representations.

To evaluate feature extraction performance, the performance between raw signal and features of wavelet coefficients was compared. In the comparison configuration, if input features are wavelet coefficients, the learning output will be wavelet coefficients transformed from the hemodynamics parameter signal; if input features are raw signals, the model will predict the same hemodynamics parameter waveform signal directly. The wavelet coefficient matrix will be directly fed into a neural network model that can take multi-dimensional input. In contrast, the wavelet coefficient matrix will be converted into a long vector to provide the other AI models that only can take two-dimensional input.

Convolutional Neural Network with Residual Block Structure

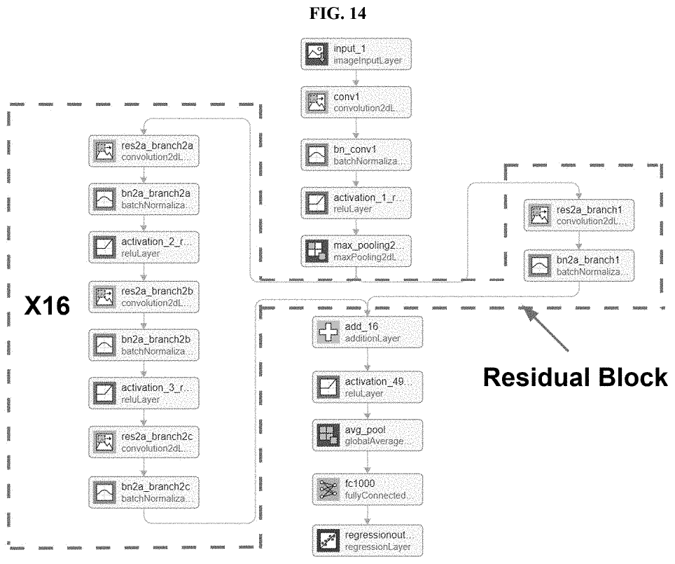

The residual neural network was initially proposed to solve classification problems, especially for image classification and segmentation tasks. One big problem of a deep network is the vanishing gradient problem. The deeper network is, the harder it is to train. In the residual neural network (e.g., in FIG. 14), the output from the previous layer, called residual, is added to the production of the current layer. Therefore, the vital information was carried from top to bottom, which addressed the gradient vanishing problem. The loss function is the mean square error in this example since the models conducted a regression task to predict continuous outcomes.

Statistical Analysis

For the continuous variables, the mean and standard deviation were calculated. For count variables, frequency counts and percentages were calculated. A two-sample test for proportions and a Fisher's exact test were adopted to test the difference of genders and the number of mortalities between training and test groups. A two-sample t-test was also used to test equal means of hospital stay time, ICU stay time, and waveform record time between training and testing cohorts. Statistical optimization of the gradient boosting tree model was done through iterative training using the XGBoost package. The following measures of diagnostic performance were formally analyzed, including R² score, the mean of square error, mean of absolute error, mean of absolute percentage error, and explained variance score. A two-sided 95% CI summarizes the sample variability in the estimates. CIs for the above measures were obtained by the bootstrap method with 20,000 replications.

### Example 2. Evaluation of AI Models and Prediction of ABP Based on ECG and PPG

180 patients of Example 1 were selected. The ECG and PPG waveform records were fed into the model of CNN with residual structure. The waveform records were processed according to Example 1: waveform sampling, data segmentation, feature extraction, model tuning and comparison, and subsequent model evaluation. Arterial blood pressure (ABP) was predicted using the AI models and the performance of the AI models is summarized in Table 1 presented below.

TABLE 1
The Prediction Performance Comparison with 95% CI.

| Model | R² | MSE | MAE | MAPE | EV score |
|---|---|---|---|---|---|
| ResNet + | 88.05% | 25.79 | 3.69 | 0.0495 | 88.06% |
| Wavelet | (85.32- | (21.33- | (3.06- | (0.031- | (86.2- |
| scatter | 94.12) | 27.65) | 4.2) | 0.0508) | 94.55) |
| transform | | | | | |
| features | | | | | |

Mean of square error (MSE); mean of absolute error (MAE); mean of absolute percentage error (MAPE); explained variance score (EV score).

### Example 3. Evaluation of AI Models and Prediction of PAP Based on ECG, PPG and Respiration Waveforms

180 patients of Example 1 were selected. The ECG, PPG and respiration waveform records were fed into the model of CNN with residual structure. The waveform records were processed according to Example 1: waveform sampling, data segmentation, the feature extraction, model tuning and comparison, and subsequent model evaluation. Pulmonary blood pressure (PAP) was predicted using the AI models and the performance of the AI models is summarized in Table 2 presented below.

TABLE 2
The Prediction Performance Comparison with 95% CI.

| Model | R² | MSE | MAE | MAPE | EV score |
|---|---|---|---|---|---|
| ResNet + | 78.82% | 12.2 | 6.62 | 0.132 | 80.19% |
| Wavelet | (55.31- | (10.92- | (6.06- | (0.109- | (78.29- |
| scatter | 86.01) | 12.9) | 7.19) | 0.148) | 82.53) |
| transform | | | | | |
| features | | | | | |

Mean of square error (MSE); mean of absolute error (MAE); mean of absolute percentage error (MAPE); explained variance score (EV score).

### Example 4. Evaluation of AI Models and Prediction of CVP Based on ECG, PPG and Respiration Waveforms

180 patients of Example 1 were selected. The ECG, PPG and respiration waveform records were fed into the model of CNN with residual structure. The waveform records were processed according to Example 1: waveform sampling, data segmentation, the feature extraction, model tuning and comparison, and subsequent model evaluation. Central venous pressure (CVP) was predicted using the AI models and the performance of the AI models is summarized in Table 3 presented below.

TABLE 3
The Prediction Performance Comparison with 95% CI.

| Model | R² | MSE | MAE | MAPE | EV score |
|---|---|---|---|---|---|
| ResNet + | 88.11% | 3.06 | 1.28 | 0.4139 | 88.73% |
| Wavelet | (86.61- | (2.92- | (1.16- | (0.399- | (86.59- |
| scatter | 92.01) | 3.13) | 1.29) | 0.438) | 92.63) |
| transform | | | | | |
| features | | | | | |

Mean of square error (MSE); mean of absolute error (MAE); mean of absolute percentage error (MAPE); explained variance score (EV score).

### Example 5. Evaluation of AI Models and Prediction of PAP Based on ECG, PPG Respiration Waveforms, Synthesized ABP and Synthesized CVP

180 patients of Example 1 were selected. The ECG, PPG and respiration waveform records and synthesized ABP and synthesized CVP waveforms were fed into the model of CNN with residual structure. The synthesized ABP were the outputs obtained from ABP prediction model described in Example 2. The synthesized CVPs were the outputs from CVP prediction model describe in Example 4.

The waveform records were processed according to Example 1: waveform sampling, data segmentation, the feature extraction, model tuning and comparison, and subsequent model evaluation. Central venous pressure (CVP) was predicted using the AI models and the performance of the AI models is summarized in Table 4 presented below.

TABLE 4
The Prediction Performance Comparison with 95% CI.

| Model | R² | MSE | MAE | MAPE | EV score |
|---|---|---|---|---|---|
| ResNet + | 86.88% | 7.52 | 3.14 | 0.096 | 87.11% |
| Wavelet | (84.36- | (7.12- | (3.01- | (0.089- | (85.88- |
| scatter | 89.01) | 7.71) | 3.25) | 0.101) | 89.89) |
| transform | | | | | |
| features | | | | | |

Mean of square error (MSE); mean of absolute error (MAE); mean of absolute percentage error (MAPE); explained variance score (EV score).

### Example 6. Evaluation of AI Models and Prediction of PAP Based on ABP, CVP, RESP, PPG, and ECG

180 patients of Example 1 who underwent PAC were selected. Patient demographic and clinical characteristics were analyzed and the data are shown in Table 5.

TABLE 5
Summary Statistics of Demographic Data and Clinical
Characteristics of All 180 Patients.

| | | Training + | | |
|---|---|---|---|---|
| | Total | Validation | Testing | P-value |
| Age, Mean ± | 63.06 ± | 61.21 ± | 63.15 ± | <0.0001 |
| SD, year | 14.57 | 13.44 | 15.21 | |
| Male, n (%) | 118 | 60 | 58 | <0.0001 |
| | (66.56) | (33.33) | (32.22) | |
| Hospital stay time, | 950.39 ± | 921.39 ± | 985.9 ± | 0.25 |
| Mean ± SD, hour | 1954.36 | 1333.39 | 1900.06 | |
| ICU stay time, | 11.62 ± | 15.3 ± | 8.22 ± | 0.63 |
| Mean ± SD, hour | 15.97 | 20.35 | 19.99 | |
| Waveform record time, | 2.07 ± | 4.51 ± | 3.01 ± | 0.72 |
| Mean ± SD, hour | 2.97 | 6.32 | 4.38 | |
| Mortality, n (%) | 37 | 20 | 17 | <0.0001 |
| | (20.56) | (11.11) | (9.44) | |

Standard deviation (SD); intensive care unit (ICU).

The distributions of these background characteristics in the training and testing groups and listed the associated p-values in the table. Moreover, the ethnicity, religion, ICU unit, marital status, insurance, language, and ICD 9 diagnosis code are presented in FIG. 14A total of 924 ICD-9 diagnosis codes are given when patients are discharged from ICU or died. The FIG. 14G indicates that the top 3 reasons for these 180 patients' admission into ICU are hypertension, heart failure, and coronary heart disease.

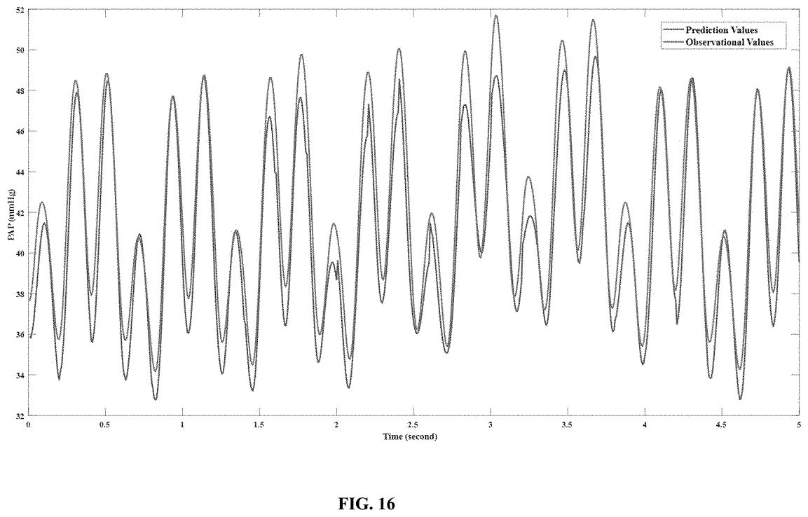

A total of 49 models with different learning parameters, feature extraction methods, sampling parameters (shown in FIG. 11.) were compared. The comparison result shows that the residual convolutional neural network consisted of 50 convolutional layers, the window size of 1 second, step size of 0.2 seconds (presented in FIG. 12B.), and wavelet scatter transform features attained the highest R² score of 96.89% and achieved the following performance metrics and 95% CIs, mean of square error of 2.52 (1.62-2.63), mean of absolute error of 1.14 (1.06-1.19), mean of absolute percentage error of 0.043 (0.029-0.048), and explained variance score of 97.11 (96.29-98.53). A sample of prediction PAP waveform with observational values was presented in FIG. 16.

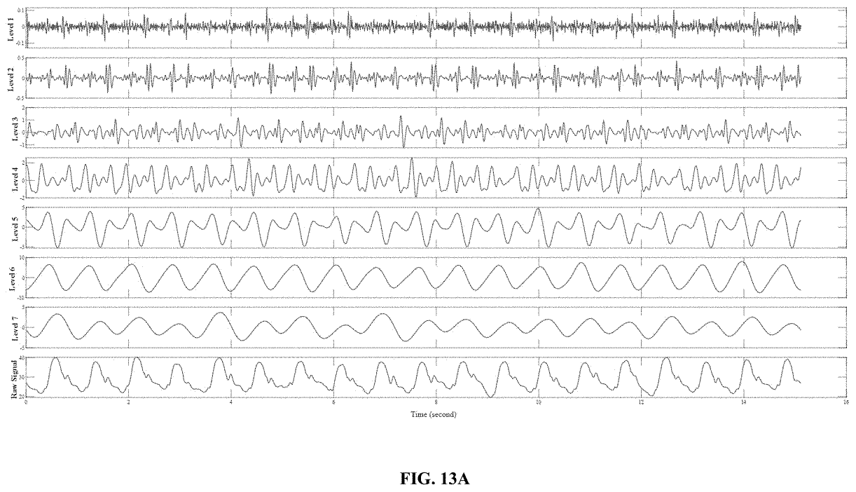

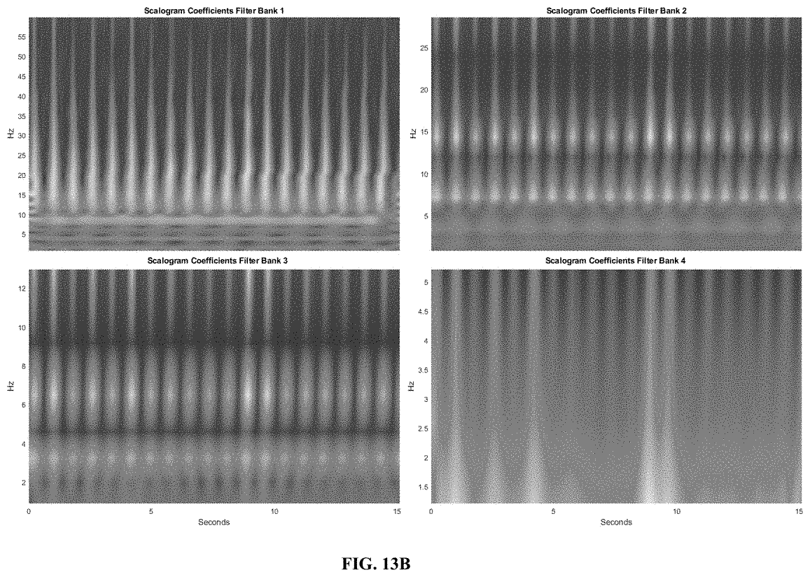

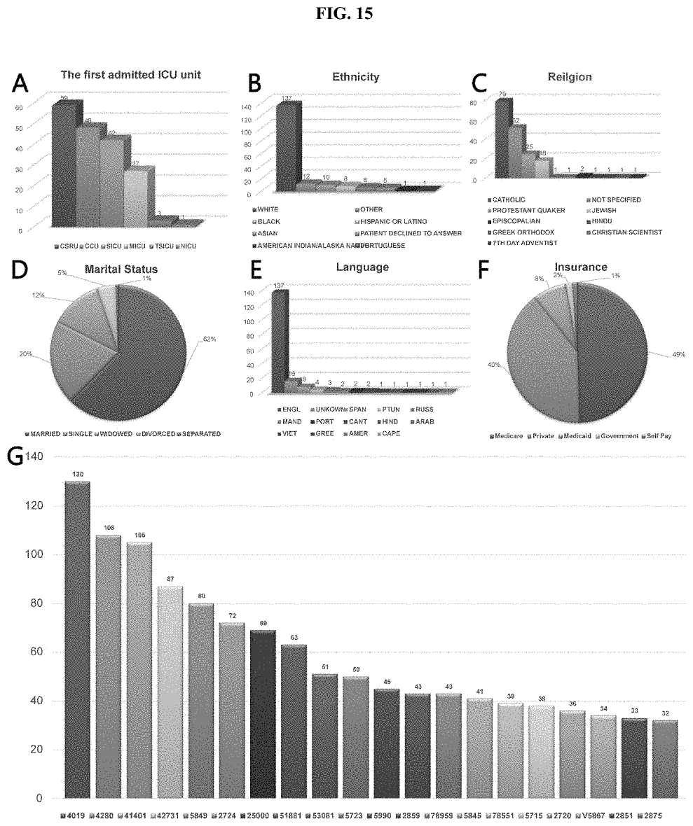

In this example, the AI model that achieved the highest R² score adopted the resNet50 design structure (shown in FIG. 15) fed by wavelet scattering features, representing 50 convolutional layers embedded in network architecture. A maximal overlap discrete symlet4 wavelet transform of a PAP signal was shown in FIG. 13A. In FIG. 13A, a segment of output signal PAP was decomposed to seven level components by a symlet4 wavelet function, representing the frequency from high to low. In order to depressed noise and yield smooth output waveform, the output of AI model are coefficients of wavelet decomposition. So that in real application, after AI model gave the predictions of wavelet coefficients one program will convert them back to waveform data. Therefore, clinical physicians can interpret mean, systolic, or diastolic blood pressure. As such, FIG. 13A presents the waveforms constructed by different level wavelet coefficients. The network input shape is 5*1008*4, 5 signals, 1008 features, 4 channels (resolution scales defined in wavelet scatter transformation). Each input sample that consisted of five signals with 2 seconds window length will be transferred to a coefficient matrix with a size of 5*1008*4, which means each signal was decomposed into four spectrum pictures (Shown in FIG. 13B). In FIG. 13B, four spectrograms present a segment of ECG signals after wavelet scattering transformation in which the four filter banks and the Gabor wavelet function were used. Each input signal of AI model, such as ECG, will pass wavelet scattering transformation and give out the coefficients of transformation. These coefficients will be used as input of the AI model. As such, FIG. 13B shows the power spectrum of 4 level wavelet scattering transformation coefficients. The output is a linear layer with 875 nodes (125*7), a vector of wavelet coefficients transformed from a segment of PAP single with a window size of 1 second. Table 6 shows that the performance of wavelet transform feature extraction exceeded that of raw signals on every performance measurement aspect.

TABLE 6
The Prediction Performance Comparison with 95% CI.

| | R² | MSE | MAE | MAPE | EV score |
|---|---|---|---|---|---|
| ResNet + | 96.89% | 2.52 | 1.14 | 0.043 | 97.11% |
| Wavelet | (95.36- | (1.42- | (1.06- | (0.029- | (96.29- |
| scatter | 99.01) | 2.63) | 1.19) | 0.048) | 98.53) |
| transform | | | | | |
| features | | | | | |
| ResNet + | 91.62% | 12.6 | 5.31 | 0.116 | 90.86% |
| Raw signal | (89.78- | (8.64- | (3.86- | (0.62- | (80.35- |
| | 93) | 18.99) | 9.45) | 0.186) | 98.85) |

Mean of square error (MSE); mean of absolute error (MAE); mean of absolute percentage error (MAPE); explained variance score (EV score).

What is claimed is:

1. An apparatus for predicting a hemodynamics parameter being conventionally obtained from an implanted sensor or catheter, comprising:
a processor configured to perform a computer program to
choose a qualified waveform record of a noninvasive input variable being collected by a noninvasive sensor or detector;
analyze the waveform record with an AI model; and
predict the hemodynamics parameter.

2. The apparatus of claim 1, wherein the noninvasive input variable is selected from a noninvasive biosignal/a noninvasive hemodynamics parameter, a general input variable, or a combination thereof.

3. The apparatus of claim 2, wherein the noninvasive input variable contains at least two noninvasive biosignals or noninvasive hemodynamics parameters selected from a group consisting of electrocardiographic (ECG), impedance cardio graphic (ICG), phonocardiogram (PCG), pulse oximetry plethysmograph (PPG), peripheral venous pressure waveform (PVPW), peripheral arterial pressure waveform (PAPW), respiration waveform (RESP), echocardiogram, airway resistance, blood sugar level waveform, and a combination thereof.

4. The apparatus of claim 2, wherein the general input is selected from the group consisting of null, age, gender, body mass index (BMI), temperature, motion status, and a combination thereof.

5. The apparatus of claim 1, wherein the AI model is a base regression model, Bagging regressor, AdaBoost regressor, voting regressor, or a combination thereof.

6. The apparatus of claim 5, wherein the base regression model is selected from a group consisting of Decision Tree (DT), K Nearest Neighbors (KNN), Nearest Centroid (NC), Gaussian Naive Bayesian (GNB), Multinomial Naive Bayesian (MNB), Complement Naive Bayesian (CNB), Bernoulli Naive Bayesian (BNB), General Linear Regression (GLR), Quadratic Discriminant Analysis (QDA), Multinomial Logistic Regression (MLR), Multi-layer Perceptron Neural Net (MPN), Ridge Regression (RR), Linear Regression with Stochastic Gradient Descent (LCSGD), Passive Aggressive Regression (PAC), Linear SVC (SVC), Random Forest (RF), Extremely Randomized Trees (ERT), Gradient Boosting Tree (GBT), Extreme Gradient Boosting Tree (EGBT), convolutional neural network (CNN) with residual structure, long-term and short-term memory (LSTM) recurrent neural network, double direction LSTM, CNN with residual block and transformer structure, and a combination thereof;
wherein Bagging regressor, if present, is a meta-regressor and uses all of the base regression models;
wherein AdaBoost regressor, if present, is a meta-estimator and utilizes the base models of DT, GNB, MNB, CNB, BNB, MLR, RR, LCSGD, and SVC; and
wherein Voting regressor, if present, is a meta-estimator and uses the base models of DT, KNN, GNB, MNG, CNB, BNB, GLR, MLR, QDA, RR, LCSGD, PAC, MPN, SVC, RF, ERT, and GBT.

7. The apparatus of claim 5, wherein the AI model is convolutional neural network (CNN) with residual structure, long-term and short-term memory (LSTM) recurrent neural network, double direction LSTM, CNN with residual block and transformer structure, EGBT, or a combination thereof.

8. The apparatus of claim 1, wherein the waveform record is segmented to multiple sample windows and the window size is from about 0.5 second to about 5 seconds with a space of about 0.5 seconds, and the step size is from about 0.1 second to the value of the window size with a span of about 0.1 second.

9. The apparatus of claim 1, wherein the hemodynamics parameter is selected from a group consisting of pulmonary arterial pressure (PAP), pulmonary arterial widget pressure (PAWP), arterial blood pressure (ABP), central venous pressure (CVP), right atrial pressure (RAP), right ventricular pressure (RVP), cardiac output (CO), stroke volume (SV), left ventricular ejection fraction (LVEF), and a combination thereof.

10. The apparatus of claim 9, wherein the hemodynamics parameter is pulmonary artery pressure (PAP).

11. A computer implemented method of predicting a hemodynamics parameter being conventionally obtained from an implanted sensor or catheter apparatus in the mammal, comprising:
choosing a qualified waveform record of a noninvasive input variable of a mammal being collected by a noninvasive sensor or detector;
analyzing the waveform record with an AI model; and
predicting the hemodynamic parameter.

12. The method of claim 11, wherein the noninvasive input variable is selected from a noninvasive biosignal/a noninvasive hemodynamics parameter, a general input variable, or a combination thereof.

13. The method of claim 12, wherein the noninvasive input variable contains two noninvasive biosignals or noninvasive hemodynamics parameters selected from a group consisting of electrocardiographic (ECG), impedance cardio graphic (ICG), phonocardiogram (PCG), pulse oximetry plethysmograph (PPG), peripheral venous pressure waveform (PVPW), peripheral arterial pressure waveform (PAPW), respiration waveform (RESP), echocardiogram, airway resistance, blood sugar level waveform, and a combination thereof.

14. The method of claim 12, wherein the general input is selected from the group consisting of nut body mass index (BMI), temperature, motion status, and a combination thereof.

15. The method of claim 11, wherein the AI model is a base regression model, Bagging regressor, AdaBoost regressor, voting regressor, or a combination thereof.

16. The method of claim 15, wherein the base regression model is selected from a group consisting of Decision Tree (DT), K Nearest Neighbors (KNN), Nearest Centroid (NC), Gaussian Naive Bayesian (GNB), Multinomial Naive Bayesian (MNB), Complement Naive Bayesian (CNB), Bernoulli Naive Bayesian (BNB), General Linear Regression (GLR), Quadratic Discriminant Analysis (QDA), Multinomial Logistic Regression (MLR), Multi-layer Perceptron Neural Net (MPN), Ridge Regression (RR), Linear Regression with Stochastic Gradient Descent (LCSGD), Passive Aggressive Regression (PAC), Linear SVC (SVC), Random Forest (RF), Extremely Randomized Trees (ERT), Gradient Boosting Tree (GBT), Extreme Gradient Boosting Tree (EGBT), convolutional neural network (CNN) with residual structure, long-term and short-term memory (LSTM) recurrent neural network, double direction LSTM, CNN with residual block and transformer structure, and a combination thereof;
wherein Bagging regressor, if present, is a meta-regressor and uses all of the base regression models;
wherein AdaBoost regressor, if present, is a meta-estimator and utilizes the base models of DT, GNB, MNB, CNB, BNB, MLR, RR, LCSGD, and SVC; and
wherein Voting regressor, if present, is a meta-estimator and uses the base models of DT, KNN, GNB, MNG, CNB, BNB, GLR, MLR, QDA, RR, LCSGD, PAC, MPN, SVC, RF, ERT, and GBT.

17. The method of claim 15, wherein the AI model is convolutional neural network (CNN) with residual structure, long-term and short-term memory (LSTM) recurrent neural network, double direction LSTM, CNN with residual block and transformer structure, EGBT, or a combination thereof.

18. The method of claim 11, wherein the waveform record is segmented to multiple sample windows and the window size is from about 0.5 seconds to about 5 seconds with a space of about 0.5 seconds, and the step size is from about 0.1 seconds to the value of the window size with a span of about 0.1 seconds.

19. The method of claim 11, wherein the hemodynamic parameter is selected from a group consisting of pulmonary arterial pressure (PAP), pulmonary arterial widget pressure (PAWP), arterial blood pressure (ABP), central venous pressure (CVP), cardiac output (CO), stroke volume (SV), left ventricular ejection fraction (LVEF), and a combination thereof.

20. The method of claim 19, wherein the hemodynamics parameter is pulmonary artery pressure (PAP).
